(19) United States
(12) Patent Application Publication
(10) Pub. No.: US 2013/0101967 A1
(43) Pub. Date: Apr. 25, 2013

(54) AQUEOUS COMPOSITION SUITABLE FOR INTRA-ORAL SCANNING METHODS

(71) Applicants: Ryan E. Johnson, St. Paul, MN (US); Joel D. Oxman, Minneapolis, MN (US); Brian N. Holmes, St. Paul, MN (US)

(72) Inventors: Ryan E. Johnson, St. Paul, MN (US); Joel D. Oxman, Minneapolis, MN (US); Brian N. Holmes, St. Paul, MN (US)

(73) Assignee: 3M INNOVATIVE PROPERTIES COMPANY, ST. PAUL, MN (US)

(21) Appl. No.: 13/641,899

(22) PCT Filed: Jun. 9, 2011
(86) PCT No.: PCT/US11/39726

§ 371 Date: Oct. 18, 2012

Related U.S. Application Data
(60) Provisional application No. 61/358,161, filed on Jun. 24, 2010.

(51) Int. Cl. A61K 6/00; A61B 6/14

(52) U.S. Cl. CPC A61K 6/002; A61B 6/145
USPC 433/217.1; 523/105

(57) ABSTRACT

Presently described are methods of intra-oral scanning and an aqueous dental compositions suitable for use for scanning comprising an aqueous solution of a polymer, wherein the solution increases in viscosity from ambient temperature to body temperature; and diffuse reflective particles having an average particle size of at least about 1 micron.

### BACKGROUND

In the field of digital dentistry, a (i.e. temporary) surface treatment is generally applied to the intraoral (e.g. tooth) surfaces prior to three-dimensional scanning (See for example U.S. Pat. No. 7,413,597 and US2008/0057479)

As described for example in the abstract of WO2009/150596, “aqueous imaging solutions/liquid are very difficult to completely dry.” As also described in WO2009/150596, “The underlying problem of these aqueous solutions remains that they are easily dissolved in saliva and easily removed by the tongue.” WO2009/150596 describes a method of providing a sticky coating to an intra-oral surface. Thereafter, a hydrophobic powder (comprising titanium) is sprayed onto the dried coating layer.

U.S. Pat. No. 6,854,973 describes, “a method for scanning the surface of an object in which moisture such as saliva or water is present on the surface. The method includes the step of applying a saliva and water-resistant composition to the surface, wherein the composition is characterized in that it does not readily wash off the surface after application of the composition to the surface in the presence of saliva or water. The composition forms an opaque film on the surface. The method further includes the step of scanning the surface having the film with a scanner. Several formulations for the composition are disclosed. One includes a liquid alcohol base, such as dehydrated ethyl alcohol, a reflective pigment, and a binder for promoting good adhesion of the formulation to the surface of the object being scanned. A suitable binder for scanning teeth and other oral structures is a denture adhesive such as an off-the-shelf denture adhesive, in powder form, that is mixed with the pigment and the alcohol base. Other suitable compositions can be derived by persons skilled in the art from the teachings disclosed herein.

### SUMMARY

Presently described are methods of intra-oral scanning and aqueous dental compositions suitable for use for scanning.

In one embodiment a method of intra-oral scanning is described. The method comprises applying a coating of a dental composition to an intra-oral surface or model thereof and scanning the coated surface to form a three dimension representation of the intra-oral surface. The dental composition, suitable for use in the method of intra-oral scanning, comprises an aqueous solution of a polymer, wherein the solution increases in viscosity from ambient temperature to body temperature and diffuse reflective particles.

In another embodiment, a dental composition is described. The dental composition comprises an aqueous solution of a polymer, wherein the solution increases in viscosity from ambient temperature to body temperature; and diffuse reflective particles having an average particle size of at least about 1 micron.

In each of these embodiments, the particles are preferably agglomerates of smaller (e.g. submicron particles). The smaller particles may be bonded into agglomerates with a binder. The binder is preferably water insoluble at temperatures below 40° C. Suitable binders include for example polyvinyl alcohols, cellulose ethers, polyoxyalkylene polymers, starches, sugars, and gelatin. The particles may comprise titania. The polymer of the aqueous solution preferably comprises a polyoxyalkylene block copolymer. The inclusion of such polymer can provide a reversible viscosity increase that can be reduced again to aid in removal of the aqueous composition after intra-oral scanning.

### BRIEF DESCRIPTION OF THE DRAWINGS

FIG. 1 shows a dental image capture system.

### DETAILED DESCRIPTION

Acquiring digital surface representation of intraoral structures is generally known. For example, U.S. Pat. No. 7,698,014; incorporated herein by reference, describes a method of acquiring a digital surface representation of one or more intraoral surfaces and processing the digital surface representation to obtain a three-dimensional model.

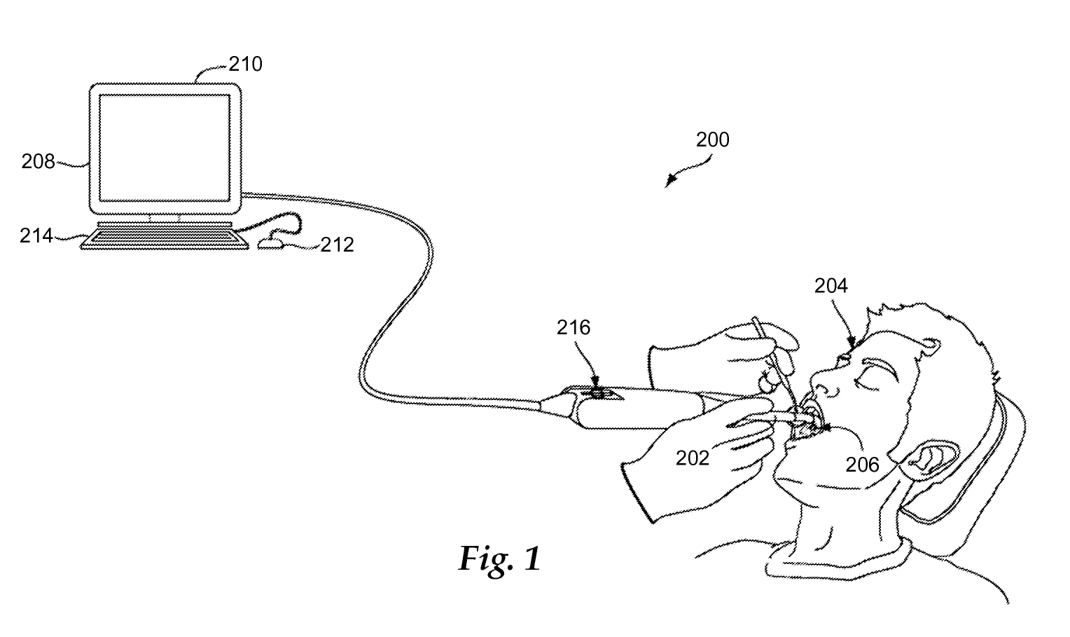

As described in U.S. Pat. No. 7,698,014, FIG. 1 shows an image capture system 200 that may include a scanner 202 that captures images from a surface 206 of a subject 204, such as a dental patient, and forwards the images to a computer 208, which may include a display 210 and one or more user input devices such as a mouse 212 or a keyboard 214. The scanner 202 may also include an input or output device 216 such as a control input (e.g., button, touchpad, thumbwheel, etc.) or a display (e.g., LCD or LED display) to provide status information.

The scanner 202 may include any camera or camera system suitable for capturing images from which a three-dimensional point cloud may be recovered. For example, the scanner 202 may employ a multi-aperture system as disclosed, for example, in U.S. Pat. Pub. No. 2004/0155975 to Hart et al. While Hart discloses one multi-aperture system, it will be appreciated that any multi-aperture system suitable for reconstructing a three-dimensional point cloud from a number of two-dimensional images may similarly be employed. In one multi-aperture embodiment, the scanner 202 may include a plurality of apertures including a center aperture positioned along a center optical axis of a lens and any associated imaging hardware. The scanner 202 may also, or instead, include a stereoscopic, triscopic or other multi-camera or other configuration in which a number of cameras or optical paths are maintained in fixed relation to one another to obtain two-dimensional images of an object from a number of slightly different perspectives. The scanner 202 may include suitable processing for deriving a three-dimensional point cloud from an image set or a number of image sets, or each two-dimensional image set may be transmitted to an external processor such as contained in the computer 208 described below. In other embodiments, the scanner 202 may employ structured light, laser scanning, direct ranging, or any other technology suitable for acquiring three-dimensional data, or two-dimensional data that can be resolved into three-dimensional data.

In one embodiment, the scanner 202 is a handheld, freely positionable probe having at least one user input device 216, such as a button, lever, dial, thumb wheel, switch, or the like, for user control of the image capture system 200 such as starting and stopping scans. In an embodiment, the scanner 202 may be shaped and sized for dental scanning. More particularly, the scanner may be shaped and sized for intraoral scanning and data capture, such as by insertion into a mouth of an imaging subject and passing over an intraoral surface 206 at a suitable distance to acquire surface data from teeth, gums, and so forth. The scanner 202 may, through such a continuous acquisition process, capture a point cloud of surface data having sufficient spatial resolution and accuracy to prepare a dental model, either directly or through a variety of intermediate processing steps.

Although not shown in FIG. 1, it will be appreciated that a number of supplemental lighting systems may be usefully employed during image capture. For example, environmental illumination may be enhanced with one or more spotlights illuminating the subject 204 to speed image acquisition and improve depth of field (or spatial resolution depth). The scanner 202 may also, or instead, include a strobe, flash, or other light source to supplement illumination of the subject 204 during image acquisition.

The computer 208 may be, for example, a personal computer or other processing device. In one embodiment, the computer 208 includes a personal computer with a dual 2.8 GHz Opteron central processing unit, 2 gigabytes of random access memory, a TYAN Thunder K8WE motherboard, and a 250 gigabyte, 10,000 rpm hard drive. This system may be operated to capture approximately 1,500 points per image set in real time using the techniques described herein, and store an aggregated point cloud of over one million points. As used herein, the term “real time” means generally with no observable latency between processing and display. In a video-based scanning system, real time more specifically refers to processing within the time between frames of video data, which may vary according to specific video technologies between about fifteen frames per second and about thirty frames per second. More generally, processing capabilities of the computer 208 may vary according to the size of the subject 204, the speed of image acquisition, and the desired spatial resolution of three-dimensional points. The computer 208 may also include peripheral devices such as a keyboard 214, display 210, and mouse 212 for user interaction with the camera system 200. The display 210 may be a touch screen display capable of receiving user input through direct, physical interaction with the display 210.

Communications between the computer 208 and the scanner 202 may use any suitable communications link including, for example, a wired connection or a wireless connection based upon, for example, IEEE 802.11 (also known as wireless Ethernet), BlueTooth, or any other suitable wireless standard using, e.g., a radio frequency, infrared, or other wireless communication medium. In medical imaging or other sensitive applications, wireless image transmission from the scanner 202 to the computer 208 may be secured. The computer 208 may generate control signals to the scanner 202 which, in addition to image acquisition commands, may include conventional camera controls such as focus or zoom.

In an example of general operation of a three-dimensional image capture system 200, the scanner 202 may acquire two-dimensional image sets at a video rate while the scanner 202 is passed over a surface of the subject. The two-dimensional image sets may be forwarded to the computer 208 for derivation of three-dimensional point clouds. The three-dimensional data for each newly acquired two-dimensional image set may be derived and fitted or “stitched” to existing three-dimensional data using a number of different techniques. Such a system employs camera motion estimation to avoid the need for independent tracking of the position of the scanner 202. One useful example of such a technique is described in commonly-owned U.S. Pat. No. 7,605,817, incorporated herein by reference. However, it will be appreciated that this example is not limiting, and that the principles described herein may be applied to a wide range of three-dimensional image capture systems.

The display 210 may include any display suitable for video or other rate rendering at a level of detail corresponding to the acquired data. Suitable displays include cathode ray tube displays, liquid crystal displays, light emitting diode displays and the like. In some embodiments, the display may include a touch screen interface using, for example capacitive, resistive, or surface acoustic wave (also referred to as dispersive signal) touch screen technologies, or any other suitable technology for sensing physical interaction with the display 210.

The digital surface representation may be processed with one or more post-processing steps. This may include a variety of data enhancement processes, quality control processes, visual inspection, and so forth. Post-processing steps may be performed at a remote post-processing center or other computer facility capable of post-processing the imaging file, which may be, for example a dental laboratory. In some cases, this post-processing may be performed by the image capture system 200. Post-processing may involve any number of clean-up steps, including the filling of holes, removing of outliers, etc.

Data enhancement may include, for example, smoothing, truncation, extrapolation, interpolation, and any other suitable processes for improving the quality of the digital surface representation or improving its suitability for an intended purpose. In addition, spatial resolution may be enhanced using various post-processing techniques. Other enhancements may include modifications to the data, such as forming the digital surface representation into a closed surface by virtually providing a base for each arch, or otherwise preparing the digital surface representation for subsequent fabrication steps.

Physical models of intraoral surfaces are also scanned for the purpose of creating a digital model or fabricating a dental articles, such as a crown.

A (i.e. temporary) surface treatment is generally applied to the intraoral (e.g. tooth) surfaces or model thereof prior to three-dimensional scanning The surface treatment comprises a particulate agent, such as titanium dioxide, to reduce the specular reflectivity, translucency and the like of the intraoral surfaces. The particles typically create a micron-scale roughness contributing to diffuse, Lambertian surface reflection characteristics.

Presently described is an aqueous dental composition that can serve as a carrier for the particulate agent. The aqueous dental composition comprises a polymer that increases the viscosity of the aqueous dental composition when the temperature is increased from ambient temperature (or lower) to body temperature. This increase in viscosity (e.g. formation of a gel) permits the composition to remain on the oral surfaces to which it was applied without being readily diluted or removed by the presence of saliva.

One class of suitable polymers that are polyoxyalkylene block copolymers, as described for example in U.S. Pat. No. 6,669,927. Such polyoxyalkylene block copolymers generally comprise terminal hydroxyl groups, more especially alpha-hydro-omega-hydroxypoly(oxyethylene) poly(oxypropylene) poly(oxyethylene) block copolymersIn general the block copolymers may be represented by the formula:

HO(CH₂H₄O)ₐ—(CH₃H₆O)_{b}(CH₄O)ₐH (Formula 1)

wherein “a” is an integer ranging from 5 to 200 and “b” is an integer ranging from 10 to 75. The average molecular weight typically ranges from about 5000 to about 20,000 g/mole.

These block copolymers, when dissolved in water or aqueous media typically form compositions which gel as their temperature is raised, but revert to liquid solutions as their temperature is lowered. In other words, the gels are reversible; cooling the gel converts the gel state to the liquid phase; whereas increasing the temperature converts the liquid phase to the gel state. The gel can be cooled down and warmed up repeatedly with no change in properties other than conversion between the gel and liquid states.

Such polyoxyalkylene block copolymers are commercially available under the trade designation “PLURONIC”. Preferred block copolymers include Pluronic F-127 and F-108. In favored embodiments, “a” of Formula 1 is at least 80, 85, 90. Further, “a” of Formula 1 is typically no greater than 160, 155, or 150. In some embodiments, “a” of Formula 1 is no greater than 135, or 130, or 125, or 120, or 115, or 110. In such favored embodiments, “b” of Formula 1 is typically at least 40 and no greater than 60. In some embodiments, “b” of Formula 1 is no greater than 50. The average molecular weight is preferably at least 10,000 g/mole, or 11,000 g/mole, or at least 12,000 g/mole. In some embodiments, the average molecular weight is less than 16,000 g/mole, or 15,000 g/mole, or 14,000 g/mole.

According to the manufacturer (BASF), Pluronic F-127 has the formula shown above wherein a is 101 and b is 56, and an average molecular weight of 12600 g/mole. Pluronic F-108 has the formula shown above wherein a is 141 and b is 44, and an average molecular weight of 14,600 g/mole. Pluronic F-127 is soluble in water, although it dissolves very slowly. Further, it is more soluble in cold than hot water.

The concentration of the block copolymers is an important parameter and can be formulated in such a manner corresponding to the other components' concentrations. By adjusting the concentration of the copolymer in the composition, any desired liquid to semi-solid transition temperature in the critical range of above ambient temperature and below body temperature can be achieved.

Although the polyoxyalkylene block copolymer Pluronics F-127 is particularly useful at concentration from about 15 wt-% to about 17 wt-%, higher molecular weight block copolymers, such as Pluronics F-108 may be used at lower concentrations of about 5 wt-% to 10 wt-%. Alternatively, lower molecular weight polyoxyalkylene block copolymer can be use at higher concentrations (e.g. ranging up to about 25 wt-%). However, such lower molecular weight polyoxyalkylene block copolymer are typically less preferred since the increase in viscosity is lower as well.

The concentration of water in the aqueous composition typically ranges from about 75 wt-% to about 95 wt-% of the composition. More typically the concentration of water is at least 75 wt-% to 85 wt-% of the composition. The water used in forming the aqueous solution is preferably purified, as by distillation, filtration, ion-exchange, or the like.

Co-solvents may be used, including anhydrous solutions comprising a polyol component such as propylene glycol or polyethylene glycol. Glycerin may also be used as a constituent of the composition.

The application temperature or pretreatment temperature is the temperature at which the composition is at the time of application to an intra-oral surface or model. The application temperature is typically about room temperature ranging from about 20° C. to about 25° C. Alternatively, the aqueous dental compositions can also be refrigerated, having a application temperature of greater than 0° C., such as about 5° C. or 10° C. provide improved stability and shelf life.

The initial viscosity of the aqueous dental composition is typically low enough such that the composition is in a liquid state. In some embodiments, the aqueous dental composition is applied at a temperature in which the composition is in a partially thickened state. In some embodiments, the initial viscosity (e.g. at 20° C. or 25° C.) as measured with a HBDV-III Cone/Plate using spindle CP40 or CP51 is less than 2,000 cps, or less than 1,000 cps, or less than 500 cps. Reducing the viscosity can aid in applying a thin (e.g. uniform) layer of the composition onto the intra-oral surfaces or model. In some embodiments, the application temperature is sufficiently low such that the aqueous composition can be applied as a rinse.

In other embodiments, the composition is applied in a partially thickened state, having a viscosity greater than 2,000 centipoise, but typically less than 6,000 centipoise, or less than 4,000 cps.

According to the manufacturer (BASF), an aqueous Pluronic F-127 solution has a low viscosity (up to a few hundred centipoise) at a temperature of about 15° C. or less for a concentration of 25 wt-%. However, the viscosity sharply rises up to about 14,000 cps as the temperature rises up to about 20° C. A 20 wt-% aqueous Pluronic F-127 solution has a viscosity near 0 centipoise at temperatures of about 19° C. or less. At 20° C. the viscosity is about 4,000 centipoise and about 6,000 centipoise. A 17 wt-% aqueous Pluronic F-127 solution has a viscosity near 0 centipoise at a temperature of about 22° C. or less. The viscosity at 30° C. increases to a maximum of about 6,000 centipoise. At concentrations of about 15 wt-%, aqueous solutions of Pluronic F-127 exhibits an increases in viscosity from about 0 centipoise at about 32° C. up to about 2,000 to 3,000 cps, at temperatures ranging from about 35° C. to 40° C. but does not necessarily form a gel.

In favored embodiments, the viscosity at body temperature (i.e. 30° C. to 40° C.) of the aqueous dental composition is at least 6,000 centipoise, or 8,000 cps, or 10,000 cps and typically no greater than about 16,000 cps.

Accordingly, the viscosity can increase by a factor of at least 2× or 3× to in excess of 16× (based on an application viscosity of about 1000 cps). Upon the lowering of the temperature, the composition preferably has the ability to reverse its viscosity and return to flow properties of a liquid. Hence, rinsing with room temperature or cooler water can cause the aqueous scanning gel to become sufficiently dilute and/or low viscosity again such that is can readily be rinsed away.

In another embodiment the initial viscosity of the composition is at a level at which the composition is in a semi-solid state at application temperature and upon exposure to a higher treatment temperature, the composition transforms into an “ultra-thick” composition or one with a substantially higher viscosity and very low flow characteristics. For compositions having initially high viscosities, the degree of thickening is typically about 2× to about 5×.

In order to be useful as a scanning gel the aqueous dental composition described herein comprises an additional component that can be detected by the imaging device. In the case of optical scanning techniques, such as the optical scanning technique previously described, the aqueous dental composition typically comprises a particulate agent that creates a micron-scale roughness contributing to diffuse, Lambertian surface reflection characteristics. Hence, such agent can be described as a diffusely reflecting particulate agent.

Although (e.g. food grade) TiO₂, has been commonly described as a component of surface treatment compositions for intra-oral scanning, various other particulate agent can be utilized provided that the material of the particulate agent sufficiently differs in refractive index from the gel. Since the gel is predominantly water, the refractive index is about 1. Provided that the material of the particulate agent differs from 1 by a refractive index of at least 0.02, such particulate agent would typically by detected by the scanning device. Suitable particulate materials include for example ZnO, ZnO₂, BaSO₄, talc, as well as various particulate food materials such as rice powder.

In addition to the refractive index property, the average particle size is also of importance for optically scanning For example, it has been found that a average particle size for optical scanners such as Lava COS (Chairside Oral Scanner, 3M ESPE, St. Paul, Minn.), is at least 1 microns and typically no greater than 30 microns or 40 microns. For scanning purposes, larger particles size of at least 5 microns, or 10 microns tend to be favored for increased scanning rates. However, particles sizes of greater than about 10 microns tend to feel gritty in the mouth. To address this problem, it is favored to employ agglomerates of smaller particles wherein the agglomerates have an average particle size of at least 10 microns, 11 microns, 12 microns, 13 microns, 14 microns, or 15 microns.

The smaller particles are typically submicron, having an average particle size of at least 0.005 microns, or 0.1 microns, or 0.2 microns.

The agglomerates are typically bonded by means of a binder. The binder is preferable insoluble in water, particularly at temperatures less than 4020 C. The insolubility of the binder insures that agglomerates do not break down into smaller particles prior to scanning Suitable binder include polyvinyl alcohols, cellulose ethers, polyoxyalkylene polymers, starches, sugars, gelatin, and surfactants such as polyoxyethylene (20) sorbitan monooleate, sorbitan mono-9-octadecenoate poly(oxy-1,2-ethanediyl), and polysorbates such as “Tween 80”.

The agglomerates are generally be rinsed away along with the aqueous dental scanning composition. Any remaining agglomerate would typically be broken down during chewing. Due to the smaller (e.g. submicron) size of the sub-particles of the agglomerates, the presence thereof generally do not feel “gritty”.

The aqueous dental compositions described herein may optionally comprise various additives are known in the art, especially flavorants and colorants. The presence of a colorant can aid in detecting that the aqueous composition has coated all the desired tooth surfaces. The intensity of a colorant can also aid in detecting the uniformity of the coating.

Various methods can be employed to apply the dental composition described herein. One method entails application of the composition to the (e.g. tooth) intra-oral surfaces to be imaged directly from the composition's container or dispenser such as a bottle, syringe, or tube. Alternatively, the composition can be applied by using a brush to paint it onto the tooth surface or by means of spraying (e.g. air-brushing). Since such application methods can be somewhat time consuming, the composition is preferably applied as a rinse.

Alternatively, the composition can be applied in a similar manner as whitening compositions by use of a dental tray or dental strips. The aqueous composition described herein is provided into a dental tray. Such dental trays can be custom fitted to a user's dentition and be made with or without reservoirs. A preferred reservoir is described in U.S. Pat. No. 6,126,443. Dental trays can be made from varying thicknesses and softness of pliable thermo-formable plastic materials. Typically, these materials are 0.02-0.08 inches thick. After dispensing the composition into the dental tray, the user then places the loaded tray into the mouth and initiates thickening of the composition. The thickening occurs when the composition is exposed to the elevated treatment temperature of the oral environment.

The dental tray may optionally be cooled to reduce the viscosity of the aqueous composition described herein or optionally heated to partially thicken the composition prior to contact with the intra-oral surfaces.

Regardless of the application techniques, the aqueous dental composition described herein generally coats all the oral (e.g. tooth) surfaces that are to be scanned. After application to intra-oral surfaces, the temperature of the aqueous composition rises to be at or near body temperature, or about 30° C. to about 40° C., causing the viscosity to increase resulting in a thickened composition.

A jet of cooled air (i.e. below body temperature) can be employed to spread the dental composition. Alternatively, heated air can accelerate the rate of thickening.

Thermally reversible dental compositions can be readily removed from the hard tissue by cooling the material below the liquid to semi-solid transition temperature, thus reversing the thickening effect. This can be accomplished with cool water or other physiologically compatible liquid. Alternatively, the concentrations of the components in the composition may be adjusted and diluted by adding water or other liquid solution. By adjusting the concentrations of the components, the transition temperature is correspondingly adjusted, and thus provides the user the ability to remove the composition even with warm solutions. Water or other liquid solutions may be administered through a rinsing cup, squirt bottle, a liquid dispensing dental tool, or any other liquid dispensing device that can provide solution to the oral environment. Preferably, administering cool or cold water provides a significant decrease in viscosity. Alternatively, the composition may be brushed, wiped, or blown off.

The aqueous dental composition and method of scanning is further illustrated by the following non-limiting examples.

Materials

TiO₂, titanium dioxide BC Purified 3328 (0.2-0.3 um), Brenntag Specialties, South Plainfield, N.J.

Zirconium dioxide powder having an average particle size of 1.6 microns, obtained from Z-Tech LLC, Bow, NH under the trade designation “CF-Plus-HM”.

PVA, polyvinyl alcohol, 98-99% Hydrolyzed PVA, Sigma-Aldrich, St. Louis, Mo.

Pluronic F127, BASF, Ludwigshafen, Germany

TiO₂ Agglomerated Particles

A 1% solution (wt/wt) of PVA in water was prepared and mixed. 50 g. of TiO₂ was mixed into 50 g. of the 1% PVA stock solution to form a slurry. The slurry was spray dried using a Buchi 290 spray dryer (Buchi Corp., New Castle, Del.), inlet temperature of 208° C., outlet temperature 80° C., with medium aspiration. The pump rate was adjusted to maintain the inlet and outlet temperatures. The dried particles were collected. The average size of the agglomerates was approximately 10-20 um.

Gel Precursor Comparative Composition—Submicron TiO₂ Particles

A 17.5% (wt/wt) solution of Pluronic F127 in deionized water was prepared. 1% (wt/wt) of unagglomerated TiO₂ particles (0.2-0.3 um) were mixed into the solution. The resulting solution was liquid at room temperature, but a gel at 37° C. This example is similar to Sample C of U.S. Pat. No. 6,669,927 except that TiO₂ particles (0.2-0.3 um mean size) were used in place of the (0.2-0.3 um mean size) fumed silica particles.

A typodont (model of the oral cavity) was warmed in a 37° C. oven. The TiO₂/Pluronic solution was brushed onto the warmed typodont and gelled quickly. The coated typodont was optically scanned according to manufacturer's instructions in a Lava COS (Chairside Oral Scanner, 3M ESPE, St. Paul, Minn.), but failed to produce a satisfactory image.

Gel Precursor Composition 1—TiO₂ Agglomerated Particles

A 20% (wt/wt) solution of Pluronic F127 in deionized water was prepared. 5% (wt/wt) of agglomerated TiO₂ particles was mixed into the solution. The resulting solution was liquid at refrigerator temperatures, but a gel at room temperature.

The cool solution was brushed onto a room temperature typodont, where it gelled. The coated typodont was optically scanned according to manufacturer's instructions in a Lava COS (Chairside Oral Scanner, 3M ESPE, St. Paul, Minn.) and produced satisfactory images.

The gelled coating could easily be removed by placing the typodont under a stream of cool water.

Gel Precursor Composition 2—1.6 Micron TiO₂ Particles

A 20% (wt/wt) solution of Pluronic F127 in deionized water was prepared. 2% (wt/wt) of the 1.6 TiO₂ particles was mixed into the solution. The resulting solution was liquid at refrigerator temperatures, but a gel at room temperature.

The cool solution was brushed onto a room temperature typodont, where it gelled. The coated typodont was optically scanned according to manufacturer's instructions in a Lava COS (Chairside Oral Scanner, 3M ESPE, St. Paul, Minn.). The scanning was slower than using Gel Precursor Composition 1, yet produced satisfactory images.

1. A method of intra-oral scanning comprising:
applying a coating of an dental composition to an intra-oral surface or model thereof wherein the dental composition comprises
an aqueous solution of a polymer, wherein the solution increases in viscosity from the application temperature to body temperature; and
diffuse reflective particles;
scanning the coated surface to form a three dimensional representation of the intra-oral surface.

2. The method of claim 1 wherein the application temperature ranges from 10° C. to 25° C.

3. The method of claim 1 wherein the aqueous solution has a viscosity of less than 1000 cps at the application temperature.

4. The method of claim 1 wherein the aqueous solution increases in viscosity by a factor of 2× to about 10×.

5. The method of claim 1 wherein the polymer is a polyoxyalkylene polymer.

6. The method of claim 1 wherein the polymer is present in the dental composition at a concentration ranging from about 10 wt-% to about 25 wt-% of the aqueous dental composition.

7. The method of claim 1 wherein the diffuse reflective particles have an average particle size of at least about 1 micron.

8. The method of claim 1 wherein the particles comprise agglomerates of smaller particles having a particles size less than 1 micron.

9. The method of claim 8 wherein the agglomerates have an average particles size of 5 to 30 microns.

10. The method of claim 8 wherein the smaller particles are bonded into agglomerates with a binder.

11. The method of claim 10 wherein the binder is water insoluble at temperatures below 40° C.

12. The method of claim 10 wherein the binder is selected from the group consisting of polyvinyl alcohols, cellulose ethers, polyoxyalkylene polymers, starches, sugars, and gelatin.

13. The method of claim 8 wherein the concentration of agglomerates ranges from about 1 wt-% to 10 wt-% at the time of application.

14. The method of claim 1 wherein the particles comprise titania.

15. An aqueous dental composition comprising:
an aqueous solution of a polymer, wherein the solution increases in viscosity from ambient temperature to body temperature; and
diffuse reflective particles having an average particle size of at least 1 micron.

16. The aqueous dental composition of claim 15 wherein the application temperature ranges from 10° C. to 25° C.

17. The aqueous dental composition of claim 15 wherein the aqueous solution has a viscosity of less than 1000 cps at the application temperature.

18. The aqueous dental composition of claim 15 wherein the aqueous solution increases in viscosity by a factor of 2× to about 10×.

19. The aqueous dental composition of claim 15 wherein the polymer is a polyoxyalkylene block copolymer.

20. The aqueous dental composition of claim 15 wherein the polymer is present in the dental composition at a concentration ranging from about 10 to 30 wt-% of the aqueous dental composition.

21-27. (canceled)
